(19) United States
(12) Patent Application Publication
(10) Pub. No.: US 2013/0178919 A1
(43) Pub. Date: Jul. 11, 2013

(54) DISPOSABLE SKIN CARE DEVICE

(71) Applicant: Andrew McNeill, St. Andrews (GB)

(72) Inventor: Andrew McNeill, St. Andrews (GB)

(21) Appl. No.: 13/811,416

(22) PCT Filed: Jun. 23, 2011
(86) PCT No.: PCT/GB2011/051181

§ 371 Date: Feb. 12, 2013

(30) Foreign Application Priority Data
Jul. 22, 2010 (GB) 1012294.3
Sep. 22, 2010 (GB) 1015849.1

(51) Int. Cl. A61N 5/06

(52) U.S. Cl. CPC A61N 5/0616
USPC 607/90

(57) ABSTRACT

There is herein described a disposable skin care device. More particularly, there is described a disposable skin care device capable of forming part of a light emitting medical apparatus.

### FIELD OF THE INVENTION

The present invention relates to a disposable skin care device. More particularly, the present invention relates to a disposable skin care device capable of forming part of a light emitting medical apparatus.

### BACKGROUND OF THE INVENTION

Ambulatory light emitting devices that are applied to the skin sit on, for example, cancerous lesions and potentially broken skin. This means that each device is contaminated after a treatment. Previously, this problem was solved in a number of ways; having a disposable device or having a device that can be sterilised.

Having a product where the entire device is sterilised is expensive, as the entire device must be constructed so that it can survive the sterilisation process. Alternatively, completely disposing of the device is expensive as the whole device can only be used once and then thrown away. Furthermore, common sterilisation processes such as autoclaving could damage the device.

It is an object of at least one aspect of the present invention to obviate or mitigate at least one or more of the aforementioned problems.

It is a further object of at least one aspect of the present invention to provide a disposable skin care device.

It is a further object of at least one aspect of the present invention to provide a disposable skin care device capable of forming part of a light emitting medical apparatus.

### SUMMARY OF THE INVENTION

According to a first aspect of the present invention there is provided a medical apparatus comprising:

a light emitting apparatus;

a light transmissive disposable device capable of being attached to the light emitting apparatus and capable of allowing light to transmit to a surface of a patient;

wherein the disposable device is capable of being removed from the light emitting apparatus after use and replaced with a replacement light transmissive disposable device.

The disposable device may be used to adhere the light emitting apparatus to the skin.

The present invention therefore resides in the provision of a disposable skin care device capable of forming part of a light emitting medical apparatus such as in ambulatory devices. The light emitting apparatus of the present invention may be sufficiently portable to enable ambulatory treatment therefore allowing treatment during which a patient may move around.

The light emitting apparatus of the present may therefore be sufficiently portable to enable ambulatory treatment therefore allowing treatment during which a patient may move around.

The light emitting apparatus may be an ambulatory device that may be capable of emitting light. Light may be emitted from a single light source or from a plurality of light sources. For example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

The light emitting apparatus may also comprise electronics to operate and control the medical apparatus along with the light source.

The light emitting apparatus may also comprise a light diffuser.

The light transmissive disposable device may be in the form of a disposable flexible substantially planar structure such as a sheet which may be flexible and/or pliable. The light transmissive disposable device may therefore be removable and may be attached to an underside of the light emitting apparatus.

The light transmissive disposable device may have a front and rear face and may comprise adhesive on at least one or both of the front and rear faces. The light transmissive disposable device may therefore be an adhesive thin sheet made from, for example, a polythene sheet with an acrylic adhesive.

The light transmissive disposable device may be attached to an underside of the light emitting apparatus using mechanical and/or chemical means such as adhesive.

The light transmissive disposable device is also intended to contact the surface of a patient and an area requiring medical treatment.

The light transmissive device may also comprise detections means to detect if the device has been used such as an electronic chip. The detection means may be located in the disposable part of the device.

The light transmissive disposable device may also be transparent (or at least substantially transparent) therefore allowing the light to be emitted from the light emitting apparatus and transmit through the light transmissive disposable device (e.g. the adhesive sheet) and then be transmitted onto the surface of the skin of a patient.

The medical apparatus may also comprise a transparent drug and/or chemical source capable of delivering drugs and/or chemicals to the area of the patient to be treated. These drugs or chemicals may be cosmetics or photosensitisers. The light emitting apparatus of the present invention may therefore be capable of performing a therapeutic and/or cosmetic treatment. The present invention may therefore relate to a light emitting apparatus capable of performing a therapeutic and/or cosmetic treatment on a human or animal patient using photodynamic therapy or phototherapy. In some situations the treatment may be said to be of therapeutic nature (e.g. skin cancer, serious acne) whereas in other situations the treatment may be said to be of cosmetic nature (e.g. minor amounts of acne, anti-aging treatments such as treatment of wrinkling).

After the light emitting apparatus has been used to treat a patient, the light transmissive disposable device (e.g. the adhesive sheet) may be removed (e.g. peeled) from the underside of the light emitting apparatus and disposed of. A replacement light transmissive disposable device (e.g. another adhesive sheet similar to the removed one) may then be placed on the underside of the light emitting apparatus. The light emitting apparatus is therefore ready to be used again. There is no need to sterilise the light emitting apparatus as this has not been in contact with a patient being treated. By disposing of the light transmissive disposable device (e.g. adhesive sheet) therefore provides an inexpensive and easy way in which to re-use the light emitting apparatus without having to go through a sterilisation process as the light transmissive disposable device (e.g. the adhesive sheet) sits between the dirty skin and clean light emitting apparatus. The light transmissive disposable device therefore functions as a removable barrier.

In particular embodiments the light emitting medical apparatus may also comprise an adhesive layer with an ambulatory light source. In these embodiments a separate cream/lotion/active may be applied separately to provide a drug and/or chemical source for a patient intended to be treated. The light emitting medical apparatus may also comprise a power source which may be small and compact and may be integrated into the whole apparatus therefore making the apparatus suitable for ambulatory treatment. The apparatus of the present invention may therefore be wearable and may be attached to a foot, leg, torso, shoulder, arm, hand, head or facial area of a patient.

The light transmissive device may also comprise a drug delivery layer capable of delivering a drug to a skin surface.

In alternative embodiments the light transmissive disposable device (e.g. the adhesive sheet) may be made from a non-transparent layer that has adhesive on at least one or both the front and rear faces of the light transmissive disposable device. To allow light to transmit to the skin surface, the light transmissive disposable device may comprise a transparent window to allow light to transmit through to the skin surface or alternatively the light transmissive disposable device may comprise a hole with no material where the light can transmit through the hole to the skin surface.

In further embodiments light transmissive disposable device (e.g. the adhesive sheet) may comprise a non-transparent layer and a transparent window (or a hole) and may only be partially coated with adhesive on the surface adjacent the underside of the light emitting apparatus. The light transmissive disposable device may therefore only be coated on the areas intended to make contact with an underside part of the light emitting apparatus. The areas where adhesive is placed on the light transmissive disposable device may therefore be adapted and configured to match or at least substantially match the shape and/or contours of the underside of the light emitting apparatus.

The light transmissive disposable device may be made from any suitable flexible material and is intended to be disposable.

In particular embodiments, the light transmissive disposable device may only comprise adhesive on the face of the light transmissive disposable device intended to engage with a skin surface.

The light transmissive disposable device may be substantially planar and/or may be substantially annular in shape. The light transmissive disposable device may therefore comprise a substantially annular surface with a substantially centrally located opening. The opening may be substantially circular in shape but can be of any suitable shape. At opposite sides of the light transmissive disposable device there may be a set of flaps. The flaps may be used to securely attach the light transmissive disposable device to a patient and can also facilitate the removal of the light transmissive disposable device from a patient and from the actual light emitting apparatus.

In particular embodiments, the light path from the light sources (e.g. LEDs or OLEDs) passes through solid material to the skin surface i.e. the light does not pass through any air or air gaps. This provides a more effective transmission of the light to skin meaning that more light intensity may enter the skin. The solid material therefore enhances light coupling from the light source to the skin. For example, a silicone gel may be used to ensure that the light travels through solid material rather than air. By providing that the light travels through solid material rather than air also facilitates the dissipation of heat and prevents heat build-up. This therefore provides heat insulation and protects the skin from any heat generated in the light source.

Typically, the light transmissive disposable device may comprise a rigid light emitter combined with a soft disposable transparent part (e.g. made from silicone gel) which can conform to curved body surfaces.

Light from light sources may also be diffused. This may be useful where there is a non-uniform light source(s) such as light from an array of LEDs. A transparent gel layer may assist in this and be used to diffuse light and therefore function as a diffuser. For example, titania nanoparticles may be added to the gel. This embodiment may therefore relate to an array of LEDs and a disposable diffusing layer which can be used to provide a more even illumination.

The light transmissive disposable device may also comprise a conductivity, fluorescence and/or oxygen sensors for the skin surface to adjust the treatment. The sensors may be in contact with the skin and may be part of the light transmissive disposable device.

The light transmissive disposable device may also be made from biocompatible material.

The light transmissive disposable device may also comprise a ‘smart’ disposable head (e.g. an electronic chip and/or sensor embedded) which allows communication between different parts of the device.

In particular embodiments, the light transmissive disposable device may be attached to a frame. Typically, the frame may be substantially annular in shape but can be of any suitable shape. The frame may comprise an outer rim and located on the rim there may be a plurality of protrusions (e.g. male features) which are intended to engage with corresponding openings (e.g. female parts) on the underside of the light emitting apparatus to which it is intended to be attached. This facilitates the correct positioning of the frame and adhesive sheet to a light emitting apparatus. The frame may also comprise a substantially centrally located opening that is intended to correspond with the opening in the light transmissive disposable device to allow light to transmit therethrough.

The light transmissive disposable device may therefore be attached to the frame using any suitable means such as adhesive (e.g. only in the areas which it makes contact with the frame). Alternatively, the light transmissive disposable device may be attached to the frame using posts that stick out from the frame and are intended to extend through corresponding apertures in the light transmissive disposable device. For example, the posts may have an ‘X’-shaped cross-section which may then be inserted through corresponding ‘X’-shaped apertures in the light transmissive disposable device. The attached light transmissive disposable device and the frame may therefore form a disposable item that may be clipped onto the underside of a light emitting apparatus using the plurality of protrusions. Once used then a new disposable item comprising a new light transmissive disposable device and frame may be attached to the underside of a light emitting apparatus.

The frame may also comprise at least one or a plurality of flexible hinges. The frame comprising at least one or a plurality of flexible hinges may be attached to a light transmissive disposable device and may therefore be capable of adapting and/or conforming to a non-planar, substantially non-planar or curved surface on, for example, any part of a human or mammal body such as on a foot, leg, torso, shoulder, arm, hand, head or facial area of a patient. By allowing the frame to adapt and/or conform to a non-planar or curved surface allows the light source to be capable of providing even or substantially even illumination of a patient's skin, which is important for efficacious therapy.

In a further alternative, the frame may be made from flexible material which allows the frame to adapt and/or conform to a non-planar, substantially non-planar or curved surface on, for example, any part of a human or mammal body such as on a foot, leg, torso, shoulder, arm, hand, head or facial area of a patient. A suitable flexible material may be an elastomeric material made from, for example, a polymeric material.

In a further alternative embodiment, the light transmissive disposable device may comprise at least one or a plurality of protruding members (e.g. posts) which may be capable of being directly attached to a light transmissive disposable device (e.g. an adhesive sheet). There may be any suitable number of protruding members. The protruding members may be attached to the light transmissive disposable device using mechanical and/or chemical means such as adhesive or an adhesive process. In these types of embodiments the light emitting apparatus may be flexible. The protruding members may also have a substantially ‘X’-shaped cross-section that fit into corresponding ‘X’-shaped apertures in the light transmissive disposable device. The light transmissive disposable device may be made from any suitable adhesive plaster material such as polyester with an acrylic adhesive.

The light emitting apparatus may contact a patient with a surface area of about 0.1-500 cm², about 0.1-250 cm², about 0.1-100 cm², about 1-100 cm² or about 5-50 cm². Alternatively, the light emitting apparatus may be capable of making contact with a patient having a surface area of greater than about 0.01 cm², greater than about 0.1 cm², greater than about 1 cm², greater than about 5 cm², greater than about 10 cm², greater than about 50 cm² or greater than about 100 cm².

The light source may be any suitable light source that may emit light over a desired wavelength. The light source may be provided as a light emitting layer.

Typically, the light source may operate within a range of about 300-3000 nm, about 300-1500 nm, about 300-800 nm or about 370-750 nm. In particular embodiments the light source may operate in the visible region of the electromagnetic spectrum. Alternatively, the light source may operate in the ultraviolet or infrared wavelength regions of the electromagnetic spectrum. The light source during operation may operate at a substantially constant and/or uniform wavelength or alternatively may be scanned over a range of wavelengths. In particular embodiments more than one wavelength of light may be emitted and/or pulsed at the same or different times. This may be suitable for the treatment of acne.

The light source may have an extensive light emitting surface area of about 0.1-500 cm², about 0.1-250 cm², about 0.1-100 cm², about 1-100 cm² or about 5-50 cm². Alternatively, the light source may have a light emitting surface of greater than about 0.01 cm², greater than about 0.1 cm², greater than about 1 cm², greater than about 5 cm², greater than about 10 cm², greater than about 50 cm² or greater than about 100 cm².

The light source may be substantially square or substantially rectangular and may have dimensions of about 0.5 cm×0.5 cm, about 1 cm×1 cm, about 2 cm×2 cm, about 5 cm×5 cm, about 10 cm×10 cm, about 20 cm×10 cm or about 15 cm×30 cm. Alternatively, the light source may be substantially circular and may have a diameter of about 0.5 cm, about 1 cm, about 2 cm, about 5 cm, about 10 cm or about 20 cm.

The light source may emit light substantially continuously over a pre-set period of time or may emit light discontinuously such as in a pulsed manner. In particular embodiments, the light from the light source may be pulsed with a period of at least about 10 ms, at least about 100 ms, at least about 1 s, at least about 10 s, at least about 10 ms, at least about 100 s, at least about 1,000 s or at least about 10,000 s.

In particular embodiments, the light source may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

The light source may have an optical power density of about 0.1-500 mW/cm², about 1-200 mW/cm² or about 5-75 mW/cm². The light source during operation may operate at a substantially constant power or alternatively may be varied over a range of powers.

The medical apparatus may comprise a photochemical and/or a photopharmaceutical preparation in the drug and/or chemical source in the form of, for example a layer.

The photochemical and/or photopharmaceutical preparation may be delivered to the area of the patient to be treated at appropriate pre-set times and/or may be controlled by a control unit. The photochemical and/or a photopharmaceutical preparation may be present in the form of, for example, a gel, ointment, cream or gauze soaked in a photosensitiser solution. Alternatively, or in addition the light emitting device may be provided with a thin film impregnated with a photochemical and/or photopharmaceutical preparation. The photochemical and/or a photopharmaceutical preparation may comprise a drug and/or antiseptic capable of treating a patient which may be applied to a person in need thereof. Typically, the photochemical and/or photopharmaceutical may be transparent or substantially transparent or may become transparent or substantially transparent during use and emission of light.

In embodiments where the photochemical and/or photopharmaceutical may be transparent or translucent to the wavelength of the irradiating light, the resulting device may be readily applied without a separate step of applying a photochemical and/or a photopharmaceutical to a patient.

Typical drugs and/or chemicals used in the present invention include but are not limited to precursor drugs such as ALA or Metfix or other photodynamic therapy agents.

In use, the drug and/or chemical layer may be placed over the top of the area of the patient to be treated. The chemical layer may be a moisturiser. The apparatus may be lightweight and portable. In particular embodiments the apparatus may be a totally self-contained portable unit and may comprise a self-contained power supply. The power supply may operate electronics in the device and the light source. The apparatus may be sufficiently portable to enable ambulatory treatment therefore allowing treatment during which a patient may move around. Treatment may therefore occur at home or at work and may be removed by a patient when necessary. This provides lower treatment costs as this avoids out-patient or in-patient stays in hospital. This provides the significant advantage that lower light levels may be used since exposure can occur for a longer period of time. This overcomes the problem of pain induced in some patients by high irradiances from conventional sources used in hospitals. Moreover, lower irradiance over a longer period of time may be more effective in photoinduced therapy as it gives more time for oxygen to diffuse to the region to be treated, and reduces photobleaching of the photopharmaceutical.

The apparatus according to the present invention may be used in a range of phototherapies and photodynamic therapies. For example, the apparatus according to the present invention may be used in the treatment of cancer (e.g. skin cancer), acne, wrinkles, wound-healing, anti-aging and post-skin laser treatments such as found in cosmetic applications. By wound is meant any form of open or closed wound. Open wounds include but are not limited to: incisions or incised wounds; lacerations; abrasions; puncture wounds; penetration wounds; gunshot wounds; and ulcers (including diabetic derived ulcers). Closed wounds include but are not limited to: contusions; hemaomas; and crushing injuries.

According to a second aspect of the present invention there is provided a method of performing a therapeutic and/or cosmetic treatment, the method comprising:

providing a light emitting apparatus;

providing a light transmissive disposable device capable of being attached to the light emitting apparatus and capable of allowing light to transmit to a surface of a patient;

wherein the disposable device is capable of being removed from the light emitting apparatus after use and replaced with a replacement light transmissive disposable device.

The method may be performed by an apparatus as defined in the first aspect.

According to a third aspect of the present invention there is provided use of an apparatus according to the first aspect in a medical treatment.

Typically, the medical treatment may be a photodynamic therapy or phototherapy.

The present invention may therefore relate to the treatment of cancer (e.g. skin cancer), acne, wrinkles, wound-healing, anti-aging and post-skin laser treatments (e.g. cosmetic applications).

According to a fourth aspect of the present invention there is provided a method of medical treatment using of an apparatus according to the first aspect.

The medical treatment may be that of photodynamic therapy or phototherapy.

Moreover, the treatment may be that of the treatment of cancer (e.g. skin cancer), acne, wrinkles, wound-healing, anti-aging and post-skin laser treatments (e.g. cosmetic applications).

### BRIEF DESCRIPTION OF THE DRAWINGS

Embodiments of the present invention will now be described, by way of example only, with reference to the accompanying drawings in which:

FIG. 1 is a side view of a schematic representation of a disposable skin care device in a light emitting medical apparatus according to an embodiment of the present invention;

FIG. 2a is a perspective view of a disposable skin care device which forms part of a light emitting medical apparatus according to an embodiment of the present invention;

FIG. 2b is a perspective view of a frame which forms part of a light emitting medical apparatus according to an embodiment of the present invention;

FIG. 3 is a perspective view of the disposable skin care device of FIG. 2a and the frame of FIG. 2b joined together to form a light emitting medical apparatus according to an embodiment of the present invention;

FIG. 4 is an exploded side view of a disposable skin care device comprising protruding elements which forms part of a light emitting medical apparatus according to an embodiment of the present invention; and

FIG. 5 is a top view of the light emitting medical apparatus of FIG. 4 which shows protruding elements fitting into the disposable skin care device.

### BRIEF DESCRIPTION

Generally speaking, the present invention resides in the provision of a disposable skin care device capable of forming part of a light emitting medical apparatus such as in ambulatory devices. The light emitting apparatus of the present invention may be sufficiently portable to enable ambulatory treatment therefore allowing treatment during which a patient may move around.

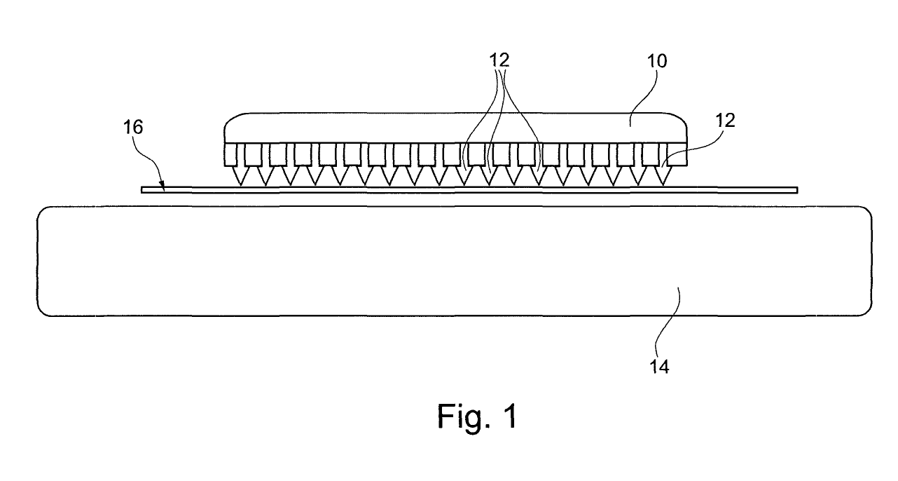

FIG. 1 represents a light emitting medical apparatus of the present invention generally designated 10. The light emitting medical apparatus 10 emits light 12 from a light source or a plurality of light sources. In particular embodiments, the light source may, for example, be any suitable form of diode such as organic light-emitting diode or an inorganic light-emitting diode incorporated into, for example, a diffuser. Alternatively, the light source may be a fluorescent light source such as a fluorescent lamp. The fluorescent light source may be compact in shape. For example, this includes technology developed from back-lit display technology, LEDs and waveguides and diffusers.

As shown in FIG. 1 there is a disposable adhesive sheet 16 located on the underside of the light emitting medical apparatus 10. The adhesive sheet 16 is flexible and/or pliable and comprises adhesive on both the front and rear faces of the sheet 16. The adhesive sheet 16 is a continuous thin layer of sheeting made from polyester with an acrylic adhesive. The sheet 16 is securely attached to the underside of the light emitting medical apparatus 10 using adhesive and is also capable of being attached to the patients skin and area to be treated. (In alternative embodiments the adhesive sheet 16 only comprises adhesive on the side adjacent the light emitting medical apparatus 10). The adhesive sheet 16 is transparent (or at least substantially transparent) therefore allowing the light 12 emitted from the light emitting medical apparatus 10, to transmit through the adhesive sheet 16 (and any adhesive) and then be transmitted onto the surface of the skin 14.

Although not shown the light emitting medical apparatus 10 may also comprise a transparent drug and/or chemical source capable of delivering drugs and/or chemicals to the area of the patient to be treated. The light emitting medical apparatus 10 of the present invention may therefore be capable of performing a therapeutic and/or cosmetic treatment. The present invention may therefore relate to a light emitting medical apparatus 10 capable of performing a therapeutic and/or cosmetic treatment on a human or animal patient using photodynamic therapy or phototherapy. In some situations the treatment may be said to be of therapeutic nature (e.g. skin cancer, serious acne) whereas in other situations the treatment may be said to be of cosmetic nature (e.g. minor amounts of acne, anti-aging treatments such as treatment of wrinkling).

After the light emitting medical apparatus 10 has been used to treat a patient, the adhesive sheet 16 may be removed (e.g. peeled) from the underside of the light emitting medical apparatus 10 and disposed of. A replacement adhesive sheet 16 may then be placed on the underside of the light emitting medical apparatus 10. The light emitting medical apparatus 10 is therefore ready to be used again. There is no need to sterilise the light emitting medical apparatus 10 as this has not been in contact with a patient being treated. By disposing of the adhesive sheet 16 therefore provides an inexpensive and easy way in which to re-use the light emitting medical apparatus 10 without having to go through a sterilisation process as the adhesive sheet 16 sits between the dirty skin and clean light emitting medical apparatus 10 and acts as a protective barrier.

Although not shown the light emitting medical apparatus 10 may also comprise a power source which is small and compact and may be integrated into the whole apparatus 10 therefore making the apparatus suitable for ambulatory treatment. The apparatus of the present invention may therefore be wearable and may be attached to a foot, leg, torso, shoulder, arm, hand, head or facial area of a patient.

In alternative embodiments the adhesive sheet 16 is made from a non-transparent layer that has adhesive on at least one or both the front and rear faces of the sheet 16. To allow light transmit to the skin surface 14, the adhesive sheet 16 comprises a transparent window to allow light to transmit through to the skin surface or the adhesive sheet 16 comprises a hole with no material where the light can transmit through the hole to the skin surface 14.

In further embodiments the adhesive sheet 16 comprises a non-transparent layer and transparent window (or a hole) and is only partially coated with adhesive on the surface adjacent the underside of the light emitting medical apparatus 10. The adhesive sheet 16 is therefore only coated in the areas intended to make contact with an underside part of the light emitting medical apparatus 10. The areas where adhesive is placed on the adhesive sheet 16 is therefore adapted and configured to match or at least substantially match the shape and/or contours of the underside of the light emitting medical apparatus 10.

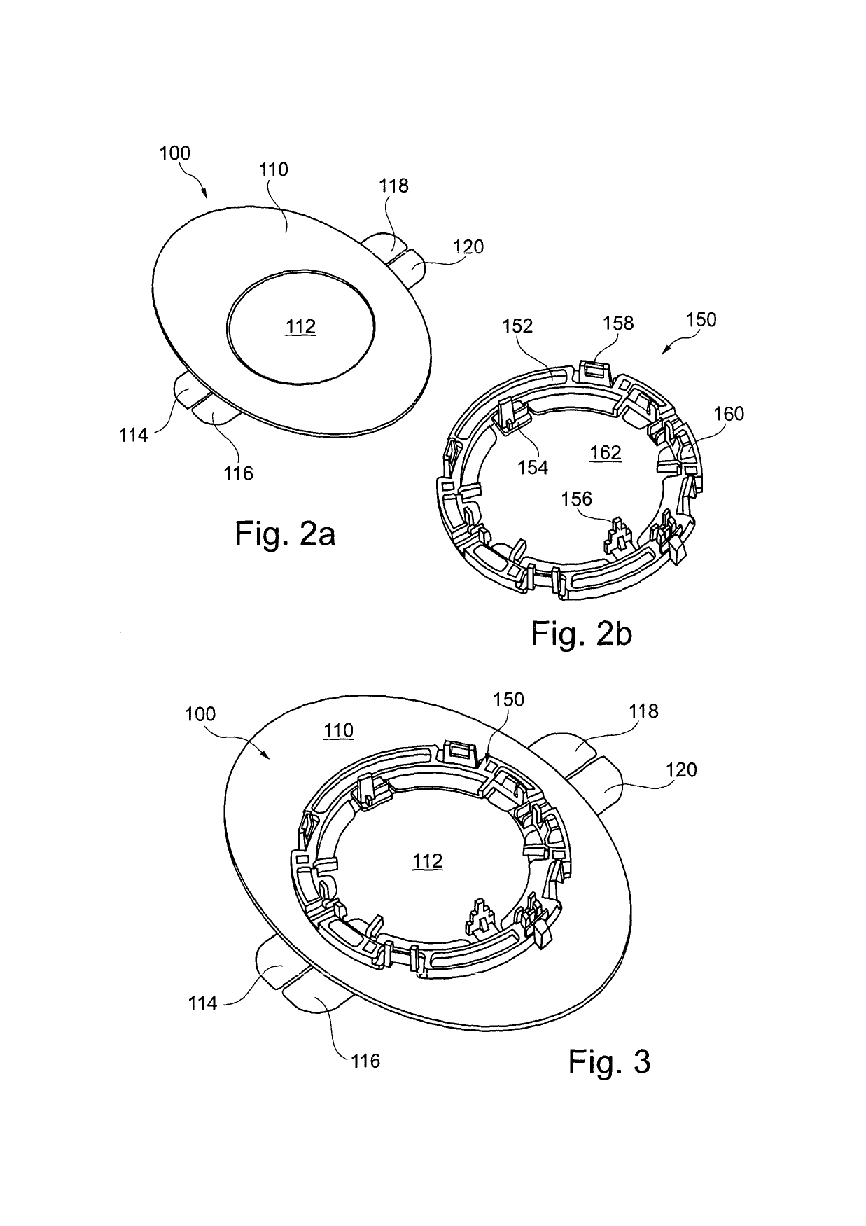

FIG. 2a is a view of an adhesive sheet 100 according to a further embodiment of the present invention. The adhesive sheet 100 is made from any suitable flexible material and is intended to be disposable. The adhesive sheet 100 only comprises adhesive on the face of the sheet 100 intended to engage with a skin surface. The adhesive sheet 100 is substantially planar and substantially annular in shape. The adhesive sheet 100 therefore comprises a substantially annular surface 110 with a substantially centrally located opening 112. The opening 112 is shown as being substantially circular in shape but can be of any suitable shape. At opposite sides of the adhesive sheet 100 there is shown a set of flaps 114,116, 118, 120. The flaps 114,116, 118, 120 can be used to securely attach the adhesive sheet 100 to a patient and can also facilitate the removal of the adhesive sheet 100 from a patient and from the light emitting apparatus.

FIG. 2b is a view of a frame 150. The frame 150 is shown as substantially annular in shape but can be of any suitable shape. The frame 150 comprises an outer rim 152. Located on the rim 152 there are a plurality of protrusions 158 (e.g. male features) which are intended to engage with corresponding openings (e.g. female parts) on the underside of the light emitting apparatus to which it is intended to be attached. This facilitates the correct positioning of the frame 150 and adhesive sheet 100 to a light emitting apparatus. The frame 150 comprises a substantially centrally located opening 162 that is intended to correspond with the opening 112 in the adhesive sheet 100.

FIG. 3 shows the adhesive sheet 100 attached to the frame 150. The adhesive sheet 100 is attached to the frame 150 using any suitable means such as adhesive (but only in the areas which it makes contact with the frame 150). Alternatively, the adhesive sheet 100 may be attached to the frame 150 using posts that stick out from the frame 150 and are intended to extend through corresponding apertures in the adhesive sheet 100. For example, the posts may have an ‘X’-shaped cross-section which are then inserted through corresponding ‘X’-shaped apertures in the adhesive sheet 100. The attached adhesive sheet 100 and the frame 150 therefore form a disposable item that may be clipped onto the underside of a light emitting apparatus using the plurality of protrusions 158. Once used then a new disposable item comprising a new adhesive sheet 100 and frame 150 may be attached to the underside of a light emitting apparatus and then also disposed of in the future after use etc.

Although not shown, the frame 150 in FIGS. 2b and 3 may comprise at least one or a plurality of flexible hinges. The frame 150 comprising at least one or a plurality of flexible hinges attached to an adhesive sheet 100 may there be capable of adapting and/or conforming to a non-planar, substantially non-planar or curved surface on, for example, any part of a human or mammal body such as on a foot, leg, torso, shoulder, arm, hand, head or facial area of a patient. By allowing the frame 150 to adapt and/or conform to a non-planar or curved surface allows the light source to be capable of providing even or substantially even illumination of a patient's skin, which is important for efficacious therapy.

In a further alternative, the frame 150 is made from flexible material which allows the frame 150 to adapt and/or conform to a non-planar, substantially non-planar or curved surface on, for example, any part of a human or mammal body such as on a foot, leg, torso, shoulder, arm, hand, head or facial area of a patient. A suitable flexible material is an elastomeric material made from, for example, a polymeric material.

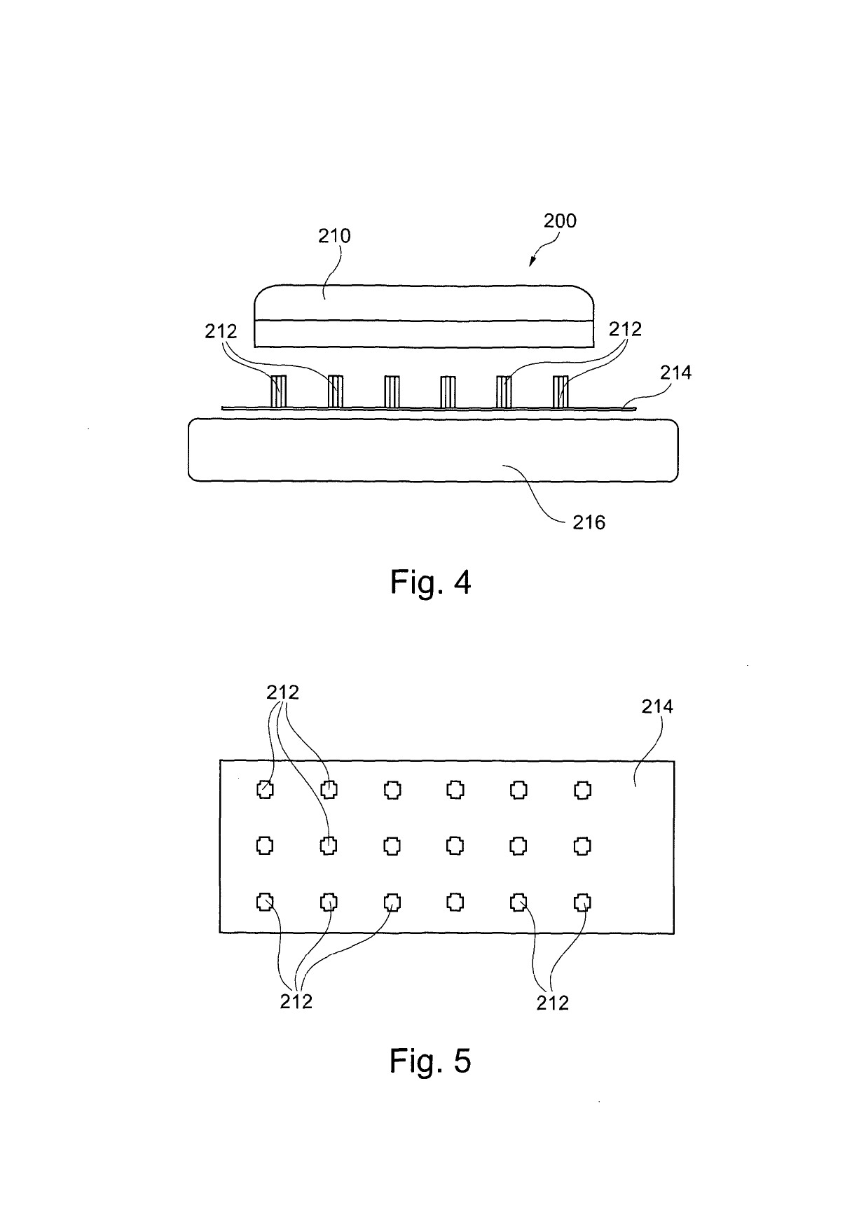

FIG. 4 is a representation of a further apparatus 200 of the present invention. The apparatus 200 comprises a light emitting apparatus 210. Shown below the light emitting apparatus 210 there is a plurality of protruding members 212 (e.g. posts) capable of being directly attached to an adhesive sheet 214. There may be any suitable number of protruding members 212. The protruding members 212 may be attached to the adhesive sheet using mechanical and/or chemical means such as adhesive or an adhesive process. In these types of embodiments the light emitting apparatus 210 may be flexible. As shown in FIG. 5 the protruding members 212 have a substantially ‘X’-shaped cross-section that fit into corresponding ‘X’-shaped apertures in the adhesive sheet 214. The adhesive sheet 214 is made from any suitable adhesive plaster material such as polyester with an acrylic adhesive.

Whilst specific embodiments of the present invention have been described above, it will be appreciated that departures from the described embodiments may still fall within the scope of the present invention. For example, any suitable type of disposable adhesive sheet may be used which may be in the form of a continuous sheet or a perforated sheet. Any type of adhesive may also be used. The disposable adhesive sheet may also be attached to any suitable type of ambulatory light emitting medical apparatus.

1. A medical apparatus comprising:
a light emitting apparatus;
a light transmissive disposable device capable of being attached to the light emitting apparatus and capable of allowing light to transmit to a surface of a patient;
wherein the disposable device is capable of being removed from the light emitting apparatus after use and replaced with a replacement light transmissive disposable device.

2. A medical apparatus according to claim 1, wherein the light transmissive device also comprises a drug delivery layer capable of delivering a drug to a skin surface; and the light transmissive device also comprises detections means to detect if the device has been used such as an electronic chip.

3 -34. (canceled)

35. A medical apparatus according to claim 1, wherein the medical apparatus is ambulatory and therefore capable of being attached to a foot, leg, torso, shoulder, arm, hand, head or facial area of a patient; and the light transmissive disposable device is in the form of a disposable flexible substantially planar structure such as a sheet which is flexible and/or pliable.

36. A medical apparatus according to claim 1, wherein the light transmissive disposable device has a front and rear face and comprises adhesive on at least one or both of the front and rear faces; and the light transmissive disposable device is in the form of an adhesive thin sheet made from, for example a polythene sheet with an acrylic adhesive.

37. A medical apparatus according to claim 1, wherein the light transmissive disposable device is transparent (or at least substantially transparent) therefore allowing the light to be emitted from the light emitting apparatus and transmit through the light transmissive disposable device (e.g. the adhesive sheet) and then be transmitted onto the surface of the skin of a patient; and the light transmissive disposable device is capable of being attached to an underside of the light emitting apparatus using mechanical and/or chemical means such as adhesive.

38. A medical apparatus according to claim 1, wherein after the light emitting apparatus has been used to treat a patient, the light transmissive disposable device (e.g. the adhesive sheet) is capable of being removed (e.g. peeled) from the underside of the light emitting apparatus and disposed of with a replacement light transmissive disposable device (e.g. another adhesive sheet) then being placed on the underside of the light emitting apparatus with the light emitting apparatus then being ready to be used again; and the light transmissive disposable device (e.g. the adhesive sheet or a short cylinder of gel) is made from a non-transparent layer that optionally has adhesive on at least one or both the front and rear faces of the light transmissive disposable device and to allow light to transmit to the skin surface, the light transmissive disposable device comprises a transparent window or hole to allow light to transmit through to the skin surface; and the light transmissive disposable device (e.g. the adhesive sheet or a short cylinder of gel) comprises a non-transparent layer and a transparent window (or a hole) and is only partially coated with adhesive on the surface adjacent the underside of the light emitting apparatus.

39. A medical apparatus according to claim 1, wherein a light path from the light sources (e.g. LEDs or OLEDs) passes through solid material (e.g. silicone gel) to the skin surface which facilitates the dissipation of heat and prevents heat build-up; and the light transmissive disposable device comprises a rigid light emitter combined with a soft disposable transparent part (e.g. made from silicone gel) which is capable of conforming to curved body surfaces.

40. A medical apparatus according to claim 1, wherein light from light sources (e.g. an array of LEDs) is diffused using a transparent gel layer which optionally may comprise titania nanoparticles; wherein the light transmissive disposable device also comprises a conductivity, fluorescence and/or oxygen sensor for the skin surface to adjust the treatment; and wherein the light transmissive disposable device is made from biocompatible material.

41. A medical apparatus according to claim 1, wherein the light transmissive disposable device also comprises a ‘smart’ disposable head (e.g. an electronic chip and/or sensor embedded) which allows communication between different parts of the device; and, wherein the light transmissive disposable device is made from a flexible material and is intended to be disposable.

42. A medical apparatus according to claim 1, wherein the light transmissive disposable device only comprises adhesive on the face of the light transmissive disposable device intended to engage with a skin surface of a patient; wherein the light transmissive disposable device is substantially planar and is substantially annular in shape and is capable of being attached to a frame comprising a plurality of protrusions (e.g. male features) which are capable of engaging with corresponding openings (e.g. female parts) on the underside of the light emitting apparatus to which it is intended to be attached.

43. A medical apparatus according to claim 42, wherein the light transmissive disposable device is attached to the frame using adhesive or using posts that stick out from the frame and are intended to extend through corresponding apertures in the light transmissive disposable device; wherein the frame comprises at least one or a plurality of flexible hinges and is therefore capable of adapting and/or conforming to a non-planar, substantially non-planar or curved surface on, for example, any part of a human or mammal body such as on a foot, leg, torso, shoulder, arm, hand, head or facial area of a patient.

44. A medical apparatus according to claim 43, wherein the frame is made from flexible material which allows the frame to adapt and/or conform to a non-planar, substantially non-planar or curved surface on, for example, any part of a human or mammal body such as on a foot, leg, torso, shoulder, arm, hand, head or facial area of a patient; and
wherein the light transmissive disposable device comprises at least one or a plurality of protruding members (e.g. posts) directly attached to the light transmissive disposable device (e.g. an adhesive sheet).

45. A medical apparatus according to claim 1, wherein the medical apparatus also comprises a transparent drug and/or chemical source capable of delivering drugs and/or chemicals to the area of the patient to be treated.

46. A medical apparatus according to claim 1, wherein the medical apparatus also comprises an adhesive layer with an ambulatory light source; and wherein a separate cream/lotion/active is applied to provide a drug and/or chemical source for a patient intended to be treated.
